(19) United States
(12) Patent Application Publication
(10) Pub. No.: US 2019/0300938 A1
(43) Pub. Date: Oct. 3, 2019

(54) CLOSTRIDIUM-SPECIFIC PROBE AND USES THEREOF

(71) Applicant: Sichuan University, Chengdu, Sichuan (CN)

(72) Inventors: Rongqing ZHOU, Chengdu (CN); Lintao HU, Chengdu (CN); Jun HUANG, Chengdu (CN); Hui LI, Chengdu (CN); Tianran BI, Chengdu (CN); Suqi CHEN, Chengdu (CN)

(21) Appl. No.: 16/240,743

(22) Filed: Jan. 5, 2019

(30) Foreign Application Priority Data
Apr. 3, 2018 (CN) 201810296666.4

(51) Int. Cl. C12Q 1/689

(52) U.S. Cl. CPC C12Q 1/689

(57) ABSTRACT

The present disclosure relates to microbiology and molecular biology, and specifically to a Clostridium-specific probe, use of the probe for detecting Clostridium content in pit mud and a method of detecting the Clostridium microorganism in fermentation pit mud using the probe. In view of the problem in the prior art that a method of detecting the number of Clostridium using fluorescence in situ hybridization has not been found, the present invention provides a complementary nucleic acid probe (SEQ ID NO: 1, named CLZ) specific to a conserved end sequence of 16S rRNA of Clostridium. The CLZ probe can be used to specifically detect Clostridium in samples such as fermentation pit mud, thereby providing an accurate and rapid analytical approach to investigate the regularity of Clostridium in a Luzhou-flavor liquor fermentation pit varying with age.

### CROSS-REFERENCE TO RELATED APPLICATION

This application claims priority to the Chinese patent application No. CN 201810296666.4, titled “Clostridium-Specific Probe and Uses Thereof”, filed on Apr. 3, 2018. The content of the aforementioned application, including any intervening amendments thereto, is incorporated herein by reference.

### TECHNICAL FIELD

The present disclosure relates to microbiology and molecular biology, and specifically to a Clostridium-specific probe, use of the probe for detecting Clostridium content in a pit mud and a method of detecting the Clostridium in a fermentation pit mud using the probe.

### BACKGROUND

Microbial community in the Luzhou-flavor liquor fermentation pit is complex and diverse, and the interaction among various communities imparts the Luzhou-flavor liquor with a unique flavor. The primary volatile component in the Luzhou-flavor liquor is esters, and among them, ethyl hexanoate is the main flavoring component of liquor. In the pit, microorganisms capable of producing volatile components such as hexanoic acid and ethyl hexanoate through metabolism play an important role in the Luzhou-flavor liquor production. Clostridium is a genus of Gram-positive bacteria which is strictly anaerobic. It is widely found in nature and also in the Luzhou-flavor liquor pit. Clostridium, including typical fermentative bacteria, is an important bacteria producing hexanoic acid. It is also an important aroma-producing functional bacterium in the fermentation of Luzhou-flavor liquor.

Therefore, the abundance of Clostridium in the pit mud and fermented grains is often used as a biological activity parameter to evaluate the quality of pit mud. However, only a small part of Clostridium strains can be screened from the pit mud using the pure culture method, which often results in a limited quantitative analysis of Clostridium, thereby failing to accurately determine the composition and content of Clostridium in the pit mud. In addition, for some uncultured Clostridia or Clostridia that grow in highly strict culture conditions, a desirable quantitative effect often cannot be reached. Therefore, there is a need to develop a more effective culture-independent quantitative analysis for Clostridia to provide a more accurate and rapid quantification of Clostridium colony in the Luzhou-flavor liquor fermentation pit.

Fluorescence in situ hybridization (FISH) is a method for specific image detection of microorganisms. A fluorescence-labeled nucleic-acid probe can not only hybridize with a nucleic acid site of high similarity to the probe in the microbial cell, but also emit fluorescence with the exciting light, thereby making it suitable for detection and positioning of microorganisms.

The method for detecting the number of Clostridia using FISH has not been developed in the prior art.

### SUMMARY

In order to solve the above problem, the present application provides a Clostridium-specific probe, a use of the probe for detecting Clostridium content in a pit mud and a method of detecting the Clostridium microorganism in a pit mud using the probe.

A Clostridium-specific probe, named CLZ, includes a nucleotide sequence shown as SEQ ID NO: 1

5′-3′: GGCTACCTTGTTACGACTTCACCCCA.

Particularly, 5′ end of the probe is further ligated with a cyanine dye fluorescein CY3.

Further, a method of preparing the probe includes steps of:

downloading full-length sequences of 16S rRNA of all microorganisms of Clostridiaceae from NCBI database;

aligning the downloaded sequences of 16S rRNA to determine an oligonucleotide sequence SEQ ID NO: 2 of intra-genus conservation and inter-genus specificity; and

designing and synthesizing the probe according to SEQ ID NO: 2

5′-3′: AGCAAACAGGATTAGATACCCTGGTAGTCCACGCCGTAAAC
GAT.

The present application further provides a use of the probe for detecting content of Clostridium.

Further, in the above use, the Clostridium includes at least one of Clostridium kluyveri, Clostridium butyricum and Clostridium perfringens.

The present application further provides a method of detecting Clostridium in a fermentation pit mud using the Clostridium-specific probe, which includes steps of:

(a) diluting a sample with a sodium pyrophosphate solution, spreading the diluted sample on a slide, keeping the slides at 35-40° C. for 1.5-2 hours, washing and air drying the slide, spreading a lysozyme solution on the dried slide, and incubating the slides for 0.5-2 hours;

(b) washing and air drying the slides obtained in step (a), dropwise adding a solution of the Clostridium-specific probe onto the slides to hybridize at 40-45° C. in the dark for 1.5-2.5 hours, washing the slides in a cleaning buffer solution preheated to 45-50° C., drying and mounting the slides with an anti-fading agent for fluorescence, and storing the slides at 20° C. in the dark; and

(c) observing the slides with a fluorescence microscope using green light as an exciting light; and calculating content of Clostridium in the sample.

In some embodiments, before diluting the sample in step (a), bacterial cells are fixed with a paraformaldehyde solution followed by centrifugation; supernatant is discarded and precipitate is dissolved with PBS at pH 7.0-8.0.

The paraformaldehyde can react with an amino of protein to coagulate the protein and PBS can also protect microbial cells, thereby making the microbial cells disperse more uniformly and easy to be counted.

Further, the centrifugation is carried out at 4° C. and 10000 rpm for 5-10 minutes.

In some embodiments, in step (a), concentration of the diluted sample is 10⁶-10⁷ cells/mL.

In some embodiments, in step (a), washing is carried out with gradient ethanol solutions at concentrations of 50%, 80% and 96%, respectively, each for 2-3 minutes.

In some embodiments, in step (a), a concentration of the lysozyme solution is 8-12 mg/mL.

In some embodiments, in step (b), the solution of the Clostridium-specific probe is prepared by mixing a 20-30 ng/μL solution of the Clostridium-specific probe with a hybridization buffer solution at a volume ratio of 1:9; and the hybridization buffer solution consists of 2.42 g/L of Tris-HCl, 1.46 g/L of EDTA, 0.1 g/L of SDS, 52.65 g/L of NaCl, 30% (v/v) of formamide and water. The hybridization buffer solution is at pH of 7.2.

In some embodiments, in step (b), the cleaning buffer solution consists of 2.42 g/L of Tris-HCl, 2.92 g/L of EDTA, 0.1 g/L of SDS, 18.02 g/L of NaCl and water. The cleaning buffer solution is at pH of 7.2.

Specifically, the above method includes the following steps.

(1) Sample Pretreatment

A sample is mixed with a 4% paraformaldehyde solution to prepare a bacterial suspension. The bacterial suspension is fixed at 4° C. overnight followed by centrifugation at 4° C. and 10000 rpm for 10 minutes. A supernatant is discarded and a precipitate is suspended with PBS at pH 7.2 to a volume of 10 mL to produce a resuspension. The resuspension is diluted with a 0.1% sodium pyrophosphate solution to a concentration of 10⁶-10⁷ cells/mL. 100 μL of the diluted resuspension is spread uniformly onto a well plate slide. The well plate slides are kept at 37° C. for 2 hours followed by washing with gradient ethanol solutions respectively having concentrations of 50%, 80% and 96% for 3 minutes. Then the well plate slides are air dried and spread with 100 μL of a 10 mg/mL lysozyme solution followed by incubation for 1 hour.

(2) Probe Hybridization

The well plate slides spread with lysozyme are washed with gradient ethanol solutions respectively having concentrations of 50%, 80% and 96% for 3 minutes. The well plate slides are air dried and dropwise added with 100 μL of a probe solution in the dark followed by hybridization at 42° C. in a humidifying box in the dark for 2 hours. The well plate slides are washed with a cleaning buffer solution preheated to 48° C. for 30 minutes and then washed with sterile water three times. The well plate slides are air dried in the dark, mounted with an anti-fading agent and stored at −20° C. in the dark.

(3) Fluorescence Microscope Observation

The mounted well plate slides are invertedly placed on a stage of the fluorescence microscope and observed using a 100× objective lens with green light (534-558 nm) as an exciting light. The content of Clostridium is obtained through calculation.

Beneficial Effects:

The application provides a Clostridium-specific probe, which is designed and synthesized based on a binding site of the Clostridium-specific probe, which is found in the 16S rRNA sequence of Clostridium. The probe has such a high specificity that it can effectively hybridize with Clostridia such as Clostridium kluyveri, Clostridium butyricum and Clostridium perfringens, thereby obtaining an accurate detection.

The application further provides a method of detecting Clostridium in the pit mud with the probe using a fluorescence in situ hybridization method. The detection method involves a simple and rapid operation and the obtained result is easy to analyze. In addition, the method of great significance can be widely applied to the detection of Clostridium in soil, water and Luzhou-flavor liquor pit mud owing to simple analysis and wide range of detection.

### BRIEF DESCRIPTION OF THE DRAWINGS

FIG. 1 shows images of microscopic morphology of Clostridium kluyveri (CICC 8022), Clostridium butyricum (CICC 10350) and Clostridium perfringens (CICC 22949, which demonstrates the specificity of CLZ probe using fluorescence in situ hybridization assay.

FIG. 2 shows microscopic morphology of Bacillus subtilis (CICC 20633) and Lactobacillus casei (ATCC 393), which demonstrates the specificity of CLZ probe using fluorescence in situ hybridization assay.

FIG. 3 is an image showing the results of detecting Clostridia in the pit mud using fluorescence in situ hybridization assay.

### DETAILED DESCRIPTION OF EMBODIMENTS

The application designs and synthesizes a Clostridium-specific probe by a fluorescence in situ hybridization method to accurately detect the content of Clostridium. The probe is designed based on the principles described as follows.

(1) A probe having a long sequence may result in an internal mismatch hybridization, so a probe having a shorter sequence may lead to a stronger specificity and the family gene can be distinguished in the hybridization. Therefore, the application employs a probe having a length of 15-45 bp and preferably 20-30 bp to ensure specificity, avoiding a stable secondary structure caused by the folding of the probe DNA.

(2) The probe of the invention has a Tₘ of 65-70° C. to ensure a preferential binding of the probe to the target fragment during the hybridization so that single hybridization signal of the probe can reflect the hybridization results.

(3) The probe of the invention has a G+C content of 40%-70% to reduce the non-specific hybridization and to ensure the hybridization specificity. In addition, the entire probe has a significantly higher content of base C than base G, thereby avoiding reduction of the hybridization efficiency.

(4) The 5′ end of the probe is labeled with a fluorescent dye Cy3 to obtain an enhanced hybridization signal derived from the fluorescence emitted by the successfully hybridized microbial cells under the exciting light.

According to the above design principles of the probe, the CLZ probe for Clostridium is synthesized and evaluated using Primer Premier 6.0. The microbial 16S rRNA sequences for the probe design are originated from the NCBI database, and the sequences are aligned using MEGA 5.2 software to select a sequence of intra-target genus conservation and inter-target genus specificity SEQ ID NO: 2 for the probe design. The probe is synthesized by Shanghai Sangon Biotech Co., Ltd., and the nucleotide sequence is shown as SEQ ID NO: 1.

The embodiments of the present invention are further explained and illustrated with reference to the following description, but are not intended to limit the scope of the present invention.

### Example 1 Synthesis of Oligonucleotide Probe

A probe was designed according to a specific sequence of Clostridium in NCBI database, and then synthesized and labeled by Shanghai Sangon Biotech Co., Ltd. The probe had a nucleotide sequence shown as SEQ ID NO: 1, and was ligated with a cyanine dye fluorescein CY3 at 5′ end thereof.

The obtained probe was dissolved in a sterile TE buffer solution to prepare a probe solution of 25 ng/μL and was stored at −20° C. The TE buffer solution was prepared by 12.11 g/L of Tris-HCl (pH 7.5) and 1.46 g/L of EDTA.

### Example 2 Use of the Probe for Detecting Clostridia in Standard Samples

(1) Preparation of Solutions

Hybridization buffer solution, comprising 2.42 g/L of Tris-HCl (pH 7.2), 1.46 g/L of EDTA, 0.01% (w/v) of SDS, 52.65 g/L of NaCl and 30% (v/v) of formamide.

Cleaning buffer solution, comprising 2.42 g/L of Tris-HCl (pH 7.2), 2.92 g/L of EDTA, 0.01% (w/v) of SDS and 18.02 g/L of NaCl.

Positive control: 5 mL of culture solutions of standard strains of Clostridium kluyveri (CICC 8022), Clostridium butyricum (CICC 10350) and Clostridium perfringens (CICC 22949) at a content of 1×10⁶ cfu/mL, respectively.

Negative control: 5 mL of culture solutions of standard strains of Bacillus subtilis (CICC 20633) and Lactobacillus casei (ATCC 393) at a content of 1×10⁶ cfu/mL, respectively.

(2) Sample Treatment

5 mL of the culture solution of the standard strain was added to a 50 mL centrifuge tube by a pipette, and was mixed uniformly with 20 mL of PBS. The centrifuge tube was centrifuged at 4° C. and 12,000 rpm to collect bacterial cells. The bacterial cells were suspended with 4% paraformaldehyde to produce a suspension. The suspension was placed at 4° C. overnight for fixation. The suspension was then centrifuged at 4° C. and 10,000 rpm for 10 minutes. Supernatant was discarded and precipitate was resuspended with PBS (pH 7.2) to a volume of 10 mL. The obtained resuspension was diluted in a gradient manner with 0.1% sodium pyrophosphate solution to a concentration of 10⁶-10⁷ cells/mL. 100 μL of the diluted resuspension was uniformly spread onto well plate slides and kept at 37° C. for 2 hours. The well plate slides were washed with gradient ethanol solutions respectively having concentrations of 50%, 80% and 96% for 3 minutes and then air dried. 100 μL of a 10 mg/mL lysozyme solution was spread onto the well plate slides followed by incubation for 1 hour.

(3) Probe Hybridization

The well plate slides spread with lysozyme was washed successively with gradient ethanol solutions respectively having concentrations of 50%, 80% and 96% for 3 minutes and was naturally air dried. 100 μL of a probe solution (which is prepared by mixing the probe in Example 1 with the hybridization buffer solution at a volume ratio of 1:9) was dropwise added on the well plate slide. The well plate slides were placed at 42° C. in a humidifying box in the dark for 2 hours for hybridization. The slides were washed in a preheated cleaning buffer solution at 48° C. for 30 minutes to remove the unhybridized probe and then washed three times with sterile water. The well plate slides were air dried in the dark and mounted with an anti-fading agent for fluorescence. The well plate slides were stored at −20° C. in the dark for observation.

(4) Observation Using Fluorescence Microscope

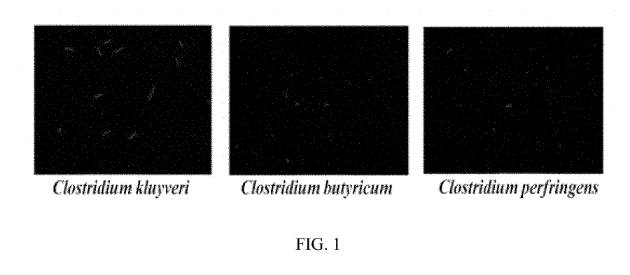

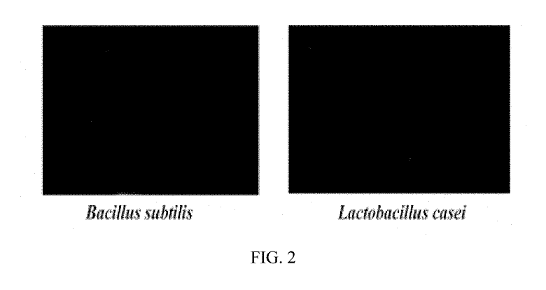

The mounted well plate slides were invertedly placed on a stage of the fluorescence microscope and observed using a 100× objective lens with green light (534-558 nm) as an exciting light. The results were shown in FIGS. 1-2.

Experimental results demonstrated that the probe CLZ can effectively hybridize with the strains selected from three species of Clostridium. This method is involves less non-specific staining, stronger fluorescence signal of a target, clear view of bacterial cells and weak background signal. The hybridization results indicated that the CLZ probe hardly hybridized with Bacillus subtilis and Lactobacillus casei, since the fluorescence emitted by the cells and clear bacterial morphologies were not observed, and only less autofluorescence from impurities in the medium was detected.

The results indicated that the CLZ probe can specifically hybridize with Clostridium, so that it is suitable for accurate detection of the Clostridium content.

### Example 3 Use of the Probe for Detecting Clostridium in Fermentation Pit Mud

(1) Preparation of Solutions

Referring to Example 2.

(2) Sample Treatment

1 g of the pit mud was mixed uniformly with 25 mL of a sterile PBS under vortex followed by centrifugation at 800 rpm. Supernatant was collected and precipitate was washed three times. All supernatants were collected and combined. The combined supernatant was centrifuged at 4° C. and 12000 rpm. Then supernatant was discarded and precipitate was resuspended with 4% paraformaldehyde to produce a resuspension. The resuspension was placed at 4° C. overnight for fixation followed by centrifugation at 4° C. and 10000 rpm. Supernatant was discarded and precipitate was resuspended with PBS (pH 7.2) to a volume of 10 mL. The obtained suspension was diluted with 0.1% sodium pyrophosphate solution in a gradient manner to concentrations of 10⁶-10⁷ cells/mL. 100 μL of the diluted suspension was uniformly spread onto well plate slides and kept at 37° C. for 2 hours. The well plate slides were washed with gradient ethanol solutions respectively having concentrations of 50%, 80% and 96% for 3 minutes, and then air dried. 100 μL of 10 mg/mL lysozyme solution was spread onto the well plate slides followed by incubation for 1 hour.

(3) Probe Hybridization

The well plate slides spread with lysozyme was washed successively with gradient ethanol solutions respectively having concentrations of 50%, 80% and 96% for 3 minutes and was naturally air dried. Then 100 μL of a CLZ probe solution (prepared by mixing the probe in Example 1 with the hybridization buffer solution in a volume ratio of 1:9) was dropwise added on the well plate slides, and 100 μL of a EUB338 probe solution (prepared by mixing a probe having a nucleotide sequence shown as SEQ ID NO: 3 with the hybridization buffer solution at a volume ratio of 1:9) was dropwise added on another well plate slide. The two well plate slides were respectively placed in a humidifying box at 42° C. in the dark for 2 hours for hybridization. The two well plate slides were washed in a preheated cleaning buffer solution at 48° C. for 30 minutes to remove the unhybridized probe and then washed three times with sterile water. The well plate slides were air dried in the dark and mounted with an anti-fading agent for fluorescence. The well plate slides were stored at −20° C. in the dark for observation.

The nucleotide sequence of the probe EUB338 (SEQ ID NO: 3) was shown as follows:

5′-3′: GCTGCCTCCCGTAGGAGT.

(4) Observation Using Fluorescence Microscope

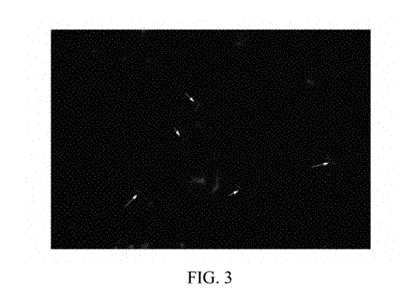

The two mounted well plate slides were invertedly placed on a stage of the fluorescence microscope and observed using a 100× objective lens with green light (534-558 nm) as an exciting light. The results were shown in FIG. 3.

5. Detection of Fermented Grains for Pit Entry and Pit Exit

Contents of total bacteria and Clostridium in the fermented grains for pit entry and pit exit were detected by repeating steps 1 to 4. The results were shown in Table 1.

TABLE 1
Contents of total bacteria and Clostridium
detected using the present method

| | | Fermented grains | Fermented grains |
|---|---|---|---|
| | Pit mud | for pit exit | for pit entry |
| Probes | (×10⁷ cells/g) | (×10⁷ cells/g) | (×10⁷ cells/g) |
| CLZ | 106 ± 9 | 0.28 ± 0.03 | 0.31 ± 0.05 |
| EUB338 | 181 ± 41 | 0.53 ± 0.03 | 0.74 ± 0.07 |

It can be seen from the results that the CLZ probe of the invention can effectively hybridize with Clostridium in the samples such as pit mud, fermented grains for pit exit and pit entry, so that it can be used for detecting the Clostridium content in such samples. In addition, the method of the invention involves an accurate detection and a wide application so that it can be used for detecting Clostridium in liquor-making industry.

1. A Clostridium-specific probe, comprising a nucleotide sequence shown as SEQ ID NO: 1.

2. The Clostridium-specific probe of claim 1, wherein 5′ end of the probe is ligated with a cyanine dye fluorescein CY3.

3. A method of preparing the probe of claim 1, comprising steps of:
downloading full-length sequences of 16S rRNA of all microorganisms of Clostridiaceae from NCBI database;
aligning the downloaded sequences of 16S rRNA to determine an oligonucleotide sequence SEQ ID NO: 2 of intra-genus conservation and inter-genus specificity; and
designing and synthesizing the probe according to SEQ ID NO: 2.

4. Use of the Clostridium-specific probe of claim 1 for detecting content of Clostridium.

5. The use of claim 4, wherein the Clostridium comprises at least one of Clostridium kluyveri, Clostridium butyricum and Clostridium perfringens.

6. A method of detecting Clostridium in a fermentation pit mud using the Clostridium-specific probe of claim 1, comprising:
(a) diluting a sample with a sodium pyrophosphate solution, spreading the diluted sample on a slide, keeping the slides at 35-40° C. for 1.5-2 hours, washing and air drying the slide, spreading a lysozyme solution on the dried slide, and incubating the slides for 0.5-2 hours;
(b) washing and air drying the slides obtained in step (a), dropwise adding a solution of the Clostridium-specific probe onto the slides to hybridize at 40-45° C. in the dark for 1.5-2.5 hours, washing the slides in a cleaning buffer solution preheated to 45-50° C., drying and mounting the slides with an anti-fading agent for fluorescence, and storing the slides at 20° C. in the dark; and
(c) observing the slides with a fluorescence microscope using green light as an exciting light, and calculating content of Clostridium in the sample.

7. The method of claim 6, comprising:
before diluting the sample in step (a), fixing bacterial cells with a paraformaldehyde followed by centrifugation, discarding supernatant and dissolving precipitate with PBS at pH 7.0-8.0.

8. The method of claim 6, wherein in step (a), a cell concentration of the diluted sample is 10⁶-10⁷ cells/mL.

9. The method of claim 6, wherein in step (b), the solution of the Clostridium-specific probe is prepared by mixing a 20-30 ng/μL solution of the Clostridium-specific probe with a hybridization buffer solution at a volume ratio of 1:9; and the hybridization buffer solution consists of 2.42 g/L of Tris-HCl, 1.46 g/L of EDTA, 0.1 g/L of SDS, 52.65 g/L of NaCl, 30% (v/v) of formamide and water.

10. The method of claim 6, wherein in step (b), the cleaning buffer solution consists of 2.42 g/L of Tris-HCl, 2.92 g/L of EDTA, 0.1 g/L of SDS, 18.02 g/L of NaCl and water.


SEQUENCE LISTING (3 sequences)

SEQ ID NO: 1 - DNA, 26 bp, Artificial Sequence
  synthetic
ggctaccttgttacgacttcacccca

SEQ ID NO: 2 - DNA, 44 bp, Artificial Sequence
  synthetic
agcaaacaggattagataccctggtagtccacgccgtaaacgat

SEQ ID NO: 3 - DNA, 18 bp, Artificial Sequence
  synthetic
gctgcctcccgtaggagt